(19) United States
(12) Patent Application Publication
(10) Pub. No.: US 2024/0124815 A1
(43) Pub. Date: Apr. 18, 2024

(54) MODULAR AND AUTONOMOUS BIOREACTOR FOR TISSUE-ENGINEERED MENISCUS CONSTRUCTS

(71) Applicant: Cornell University, Ithaca, NY (US)

(72) Inventors: Byumsu Kim, Ithaca, NY (US); Charlotte Lee, South Setauket, NY (US); Lawrence Bonassar, Ithaca, NY (US)

(21) Appl. No.: 18/484,923

(22) Filed: Oct. 11, 2023

Related U.S. Application Data
(60) Provisional application No. 63/415,605, filed on Oct. 12, 2022.

(51) Int. Cl. C12M 3/00; C12M 1/00; C12M 1/26; C12M 1/36; C12M 3/04; C12N 5/077

(52) U.S. Cl. CPC C12M 21/08; C12M 23/44; C12M 23/46; C12M 23/48; C12M 27/10; C12M 33/00; C12M 41/48; C12M 43/08; C12N 5/0655

(57) ABSTRACT

A modular bioreactor system for creating tissue-engineered constructs is described. The bioreactor system includes a bioreactor with a driving system and a chamber for holding a graft. The bioreactor system also includes a housing with a motor capable of engaging the driving system to continuously mechanically stimulate the graft.

### PRIORITY CLAIM

This application claims priority to U.S. Provisional Application Ser. No. 63/415,605, filed 12 Oct. 2022, which is expressly incorporated by reference herein.

### BACKGROUND

A tissue-engineered (TE) construct is a promising treatment for soft tissue injury in the meniscus, ligaments, tendons, neurons, etc. A bioreactor is typically used to manufacture mechanically competent engineered tissue, such as the meniscus construct, by enabling control of both environmental and mechanical conditions. It has been previously shown that tissue-engineered meniscus constructs have significantly higher mechanical properties when cultured in bioreactors under mechanical stimuli such as tensile loading. A bioreactor is required to keep a tissue-engineered construct sterile while scaling up the manufacturing process. However, typical bioreactor systems often require external power sources, making it challenging for the maintenance of the sterile environment. Furthermore, typical bioreactors are heavily customized, making the scale-up of the manufacturing process extremely expensive.

Currently, multiple types of bioreactors are used to promote cell/tissue growth, including spinner flasks, perfusion bioreactors, and mechanical stimulation bioreactors. However, to apply a mechanical force, these bioreactors require that the grafts used to create constructs be taken outside of an incubator. Alternatively, or additionally, these bioreactors require wired connections outside the incubator with a power source and controller. These requirements increase the risk of infection by exposing the grafts to external sources of infection. Alternatively, or additionally, these bioreactors are too small for the creation of tissue-engineered constructs or utilize highly customized electrical and mechanical components.

Accordingly, there is a need to develop a modular and autonomous bioreactor system that can maintain sterility, allow robust mechanical control for the creation of the tissue-engineered constructs, and increase the ability to scale up the manufacturing process of tissue-engineered constructs.

### SUMMARY

The presently disclosed bioreactor is modular, stackable, and provides mechanical stimulation to a graft to form a tissue-engineered construct. The bioreactor is mechanically stable, chemically stable, and provides a sterile environment for the creating of the tissue-engineered constructs. Thus, the bioreactor disclosed is advantageous over current bioreactors for the creation of tissue-engineered constructs.

The bioreactor includes a movable part and a fixed part, a chamber for positioning a graft for the creation of the tissue-engineered constructs, and a driving system to apply the mechanical stimulation to the graft.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIGS. 1A-1C are illustrations of a modular and autonomous bioreactor system used to create tissue-engineered constructs that can be implanted in patients;

FIG. 2 is an illustration of a bioreactor including a movable part positioned on top of a fixed part;

FIG. 3A is an illustration of a moveable part of a micrometer head-driven bioreactor that uses a micrometer head-driving system to cause a stretching function;

FIG. 3B is an illustration of a base of the micrometer head-driven bioreactor that uses a micrometer head-driving system to cause the stretching function;

FIG. 3C is an illustration of the relative location of the moveable part and the base of the micrometer head-driven bioreactor;

FIG. 4 is an illustration of a gear-rack bioreactor that uses a gear-rack driving system to cause the stretching function;

FIG. 5A is an illustration of an embodiment of a worm-gear bioreactor that uses a worm structure to cause the stretching function;

FIG. 5B is an illustration of the relative location of a moveable part and a base of the worm-gear bioreactor;

FIG. 6 is an illustration of an embodiment of the worm-gear bioreactor with an open moveable part exposing a rack;

FIG. 7A is an illustration of an embodiment of the worm-gear bioreactor with a closed moveable part;

FIG. 7B is an illustration of a self-locking snapping structure used to attach the worm structure to the base of the worm-gear bioreactor;

FIG. 7C is an illustration of a screw used to attach the worm structure to the base of the worm-gear bioreactor;

FIG. 8A is an illustration of the electronics comprised in a bioreactor system;

FIG. 8B is an illustration of the electronics protected in a housing of the bioreactor system;

FIGS. 9A-9T show images of grafts exposed to different amounts of stretching during a four (4) week of culture;

FIG. 10A shows images of the unstretched tissue-engineered construct after two (2) weeks of culture (control group);

FIG. 10B shows images of the stretched tissue-engineered construct after two (2) weeks of culture (experimental group);

FIG. 10C shows a comparison between the control and the experimental groups shown in FIGS. 10A and 10B;

FIG. 11 shows images of the unstretched tissue-engineered construct after two (2) weeks of culture (control group);

FIGS. 12A-12C shows images of stretched tissue-engineered construct after two (2) weeks of culture (experimental group);

FIG. 13 shows a histogram of collagen length after four (4) weeks of culture (red dotted line shows the initial collagen length of about 20 mm);

FIG. 14 shows brightfield images of Picrosirius red staining slides at an interface region of the unstretched control tissue-engineered constructs after two (2) weeks of culture (control group);

FIG. 15 shows brightfield images of Picrosirius red staining slides at the interface region of the stretched tissue-engineered constructs after two (2) weeks of culture (experimental group);

FIG. 16 shows brightfield images of Picrosirius red staining slides at the interface region of the unstretched tissue-engineered constructs after four (4) weeks of culture (control group);

FIGS. 17A and 17B show brightfield images of Picrosirius red staining slides at the interface region of the stretched tissue-engineered constructs after four (4) weeks of culture (experimental group);

FIGS. 18A and 18B show brightfield and polarized light images of Picrosirius red staining of the interface region of the unstretched tissue-engineered construct after two (2) weeks of culture (control group) (white arrows show the main orientation of fiber bundles);

FIGS. 19A and 19B show brightfield and polarized light images of Picrosirius red staining of the interface region of the stretched tissue-engineered construct after two (2) weeks of culture (experimental group) (white arrows show the main orientation of fiber bundles);

FIG. 20 shows polarized light images of Picrosirius red staining of the interface region of the unstretched tissue-engineered construct after four (4) weeks of culture (control group) (white arrows show the main orientation of fiber bundles);

FIGS. 21A-21C shows polarized light images of Picrosirius red staining of the interface region of the stretched tissue-engineered construct after four (4) weeks of culture (experimental group) (white arrows show the main orientation of fiber bundles);

FIG. 22 shows that a higher number of defects were observed in the collagen part of the stretched tissue-engineered constructs;

FIGS. 23A-23F show Masson's trichrome staining for cell morphology analysis at the bone, the interface, and the collagen regions of unstretched and stretched tissue-engineered construct after two (2) weeks of culture (red rectangle borders highlight the area of focused cells);

FIGS. 24A and 24B are images of the tensile testing process of the unstretched (left, FIG. 24A) and the stretched (right, FIG. 24B) tissue-engineered construct showing necking in left sample;

FIGS. 25A and 25B are images of the tensile testing process of the unstretched (left, FIG. 25A) and the stretched (right, FIG. 25B) tissue-engineered construct showing the initiation of failure in the left sample;

FIGS. 26A and 26B are images of the tensile testing process of the unstretched (left, FIG. 26A) and the stretched (right, FIG. 26B) tissue-engineered construct showing complete failure in the left sample and necking in the right sample;

FIGS. 27A and 27B are images of the tensile testing process of the unstretched (left, FIG. 27A) and the stretched (right, FIG. 27B) tissue-engineered construct showing initiation of failure in the right sample;

FIGS. 28A and 28B are images of the tensile testing process of the unstretched (left, FIG. 28A) and the stretched (right, FIG. 28B) tissue-engineered construct showing complete failure in the right sample;

FIGS. 29A-29D show bar graphs illustrating ultimate load (top left, FIG. 29A), ultimate tensile stress (bottom left, FIG. 29B), tensile modulus (top right, FIG. 29C) and strain of failure (bottom right, FIG. 29D) between unstretched (control group) and the stretched (experimental group) tissue-engineered constructs (*significant difference: p<0.05; +difference exists: 0.05<p<0.1);

FIG. 30 illustrates stress-strain curves of the tensile testing data from the unstretched (control group) and the stretched (experimental group) tissue-engineered constructs; and

FIG. 31 is a method of using a bioreactor system for creating a tissue-engineered construct.

### DETAILED DESCRIPTION

The present disclosure relates to embodiments of a bioreactor system 2 that can be used to create tissue-engineered constructs, including but not limited to ligaments, tendons, and menisci. Specifically, the present disclosure relates to embodiments of a bioreactor system 2 that can be used to provide mechanical stimulation, including but not limited to a tensile force on grafts and/or cells to form tissue-engineered constructs. The bioreactor system includes a bioreactor 10 and a housing 4 comprising a motor and a self-contained power source configured to engage the bioreactor.

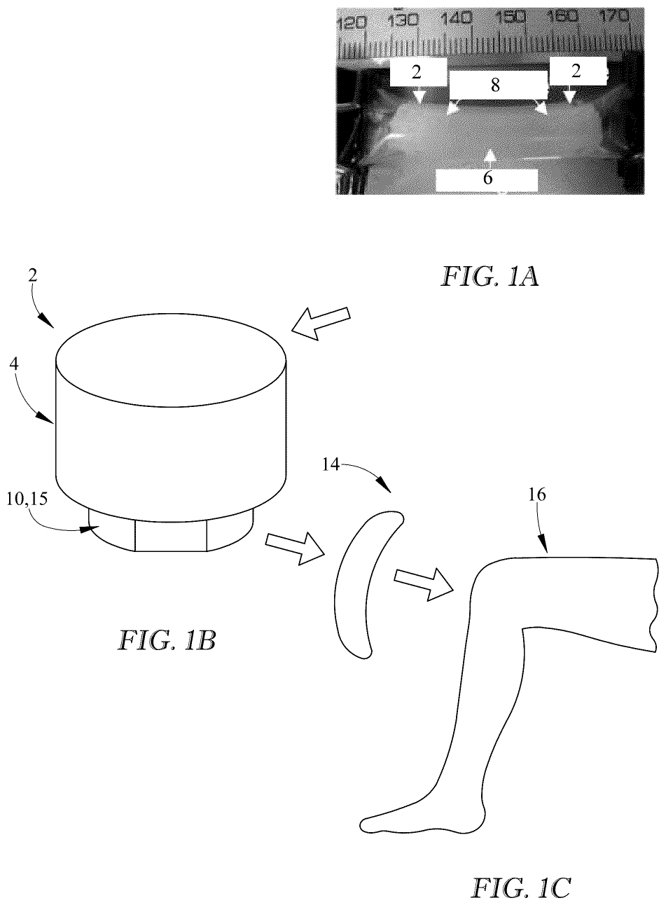

As discussed in detail herein, and shown in FIGS. 1A-1C, the bioreactor system 2 is a modular and autonomous bioreactor system 2 and can be used to provide mechanical stimulation to create a tissue-engineered construct 14 that can be implanted in a patient 16. Cells and tissues are subject to a variety of different mechanical stimulation in vivo, which has an impact on their structure, morphology, and function. The bioreactor system 2 is designed to provide a graft 12 with mechanical stimulation to mimic the in vivo loading. This mechanical stimulation can be provided by subjecting the graft 12 to a stretching function. In some embodiments, the graft 12 may be a meniscal enthesis construct comprising a bone zone 6, a collagen zone 8, and an interface region 7 between the two zones. The bioreactor system 2 provides a tunable stretching function to create a biomimetic dynamic culture to create tissue-engineered constructs 14.

The bioreactor system 2 is mechanically stable to withstand forces created during the culture process, especially during clamping and attaching the graft 12 to the bioreactor 10. The ability of the bioreactor system 2 to withstand the changing forces ensures that the graft 12 is not damaged. The bioreactor system 2 is chemically stable even after being placed in a container comprising culture media for a long time, ensuring that chemicals do not adversely affect the graft 12. The bioreactor system 2 allows the entire culture process to be carried out in a sterile environment. The bioreactor system 2 produces a stable and repeatable stretching function. The bioreactor system 2 is configured so that all measurements are accurate and any stretching length error caused by bioreactor design is minimized.

As shown in FIGS. 1A-1C, the bioreactor system 2 is modular as comprises parts that can be manufactured separately and assembled easily. For example, the bioreactor system 2 can include a stretching device or a bioreactor 10 and a housing 4. The bioreactor 10 is placed in a container with media 15. The housing 4 includes battery-powered automation and the bioreactor system 2 is configured to provide a sterile environment for the creation of the tissue-engineered constructs 14.

Certain exemplary embodiments will now be described to provide an overall understanding of the principles of the disclosure. One or more examples of these embodiments are illustrated in the accompanying drawings.

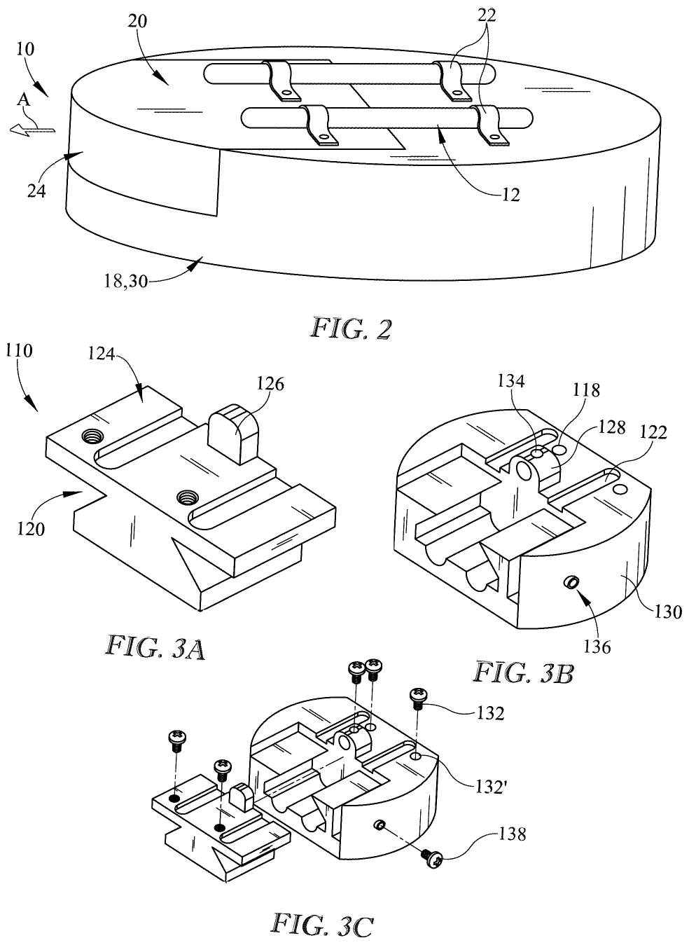

An exemplary bioreactor 10 illustrated as comprising two parts or components is shown in FIG. 2. The bioreactor 10 includes a movable part 20 positioned on top of a fixed part or a base 30. The movable part 20 includes a dovetail slide 14 that can be used to move the movable part along an axis A. The moveable part 20 includes at least two sets of clamps 22 that can be configured to hold at least two grafts 12. In other embodiments, the bioreactor 10 may include more than two sets of clamps 22. The modular bioreactor 10 is placed in a container comprising media and a driving system 18 is used to create the stretching function on the grafts 12.

In the illustrative example, shown in FIGS. 3A-3C, a micrometer head-driven bioreactor 110 uses a micrometer head driving system 118 to create the stretching function. The micrometer head-driven bioreactor 110 includes a tab 126 fixed on a slide 124, which is a movable part 120 of the micrometer head-driven bioreactor 110 (FIG. 3A, 3C). The micrometer head-driven bioreactor 110 further includes a bracket 128 fixed on a base 130 (FIG. 3B, 3C). The tab is affixed to the bracket 128 when the slide 124 is closed, i.e. when the moveable part 120 slides into the base 130. A micrometer head 134 is used as the driving system 118. At least two rectangular chambers 122 are used for the placement of two grafts 12 on each micrometer head-driven bioreactor 110. One or more screw holes 132′ are provided for screws 132 to clamp the graft 12 at its bone zones 6.

As shown in FIGS. 3A-3C, the micrometer head 134 is fixed onto the base 130 by the bracket 128 and placed horizontally above the micrometer head-driven bioreactor 110. When the micrometer head 134 is rotated, the micrometer head 134 reaches the tab 126, causing the stretching function. The slide 124 is pushed out of the base 130 as the micrometer head continues to be rotated. The stretching length can be determined through a scale on the micrometer head 134. A locking screw structure 136 is designed on the side of base 130. When the slide 124 reaches a specific position, the locking screw structure 136 is engaged to lock the slide 124 to avoid disturbance or slippage of the graft 12. In some embodiments, the micrometer head 134 can be located underneath the base 130. The micrometer head-driven bioreactor 110 can be produced with any plastic materials including but not limited to PLA and polysulfone.

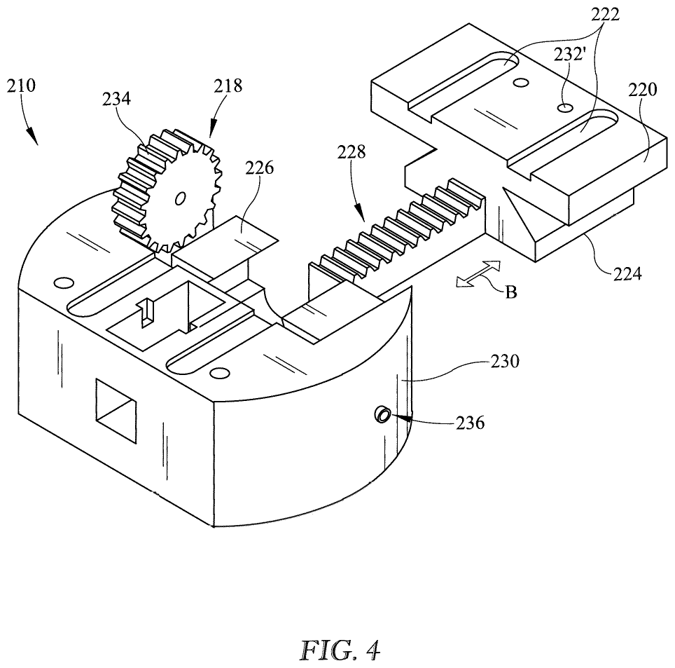

In the illustrative example, shown in FIG. 4, a gear-rack bioreactor 210 uses a gear-rack driving system 218 to cause the stretching function. A gear 234 is configured to be half buried in a base 230. The buried part of the gear 234 is configured to engage with a rack 228 inside the gear-rack bioreactor 210. The rack 228 is attached to a slide 224, which is a movable part 220 of the gear-rack bioreactor 210. The gear 234 is manually rotated, driving the rack 228 outwards along the axis B, thereby moving the slide 224. At least two rectangular chambers 222 are designed for the placement of two grafts 12 (e.g., meniscal enthesis constructs) on each gear-rack bioreactor 210. One or more screw holes 232′ are provided for screws 232 to clamp the grafts 12. When the slide 224 reaches a specific position, a locking screw structure 236 is engaged to lock the slide 224 to avoid disturbance or slippage of the graft 12. The gear-rack bioreactor 210 can be produced with any plastic materials including but not limited to PLA and polysulfone.

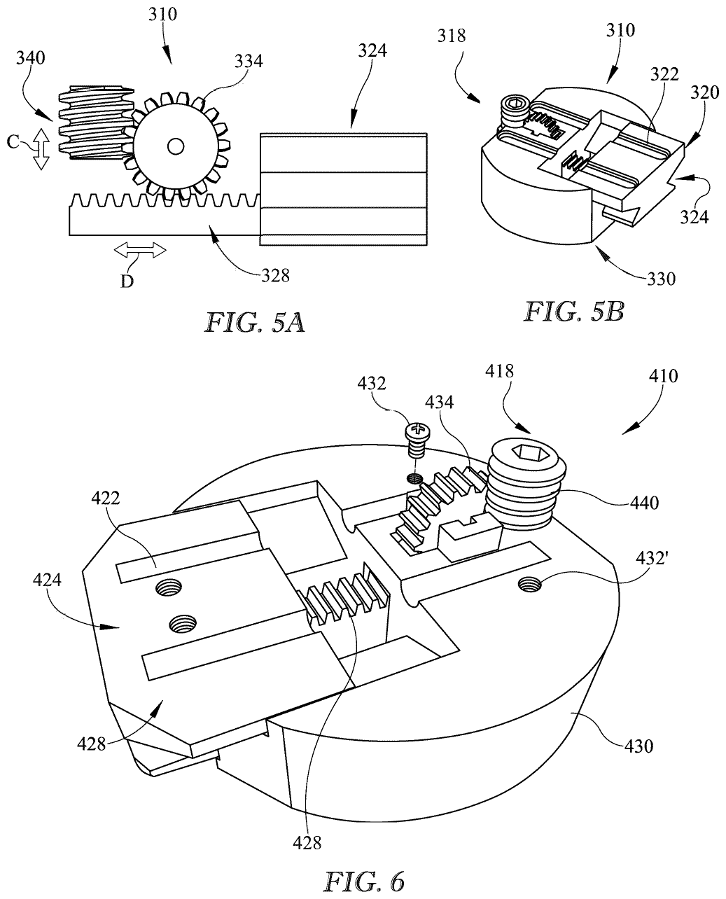

In the illustrative example, shown in FIGS. 5A and 5B, a worm-gear bioreactor 310 uses a worm structure 340 in a driving system 318 to manually drive the worm-gear bioreactor 310 to perform the stretching function. The worm-gear bioreactor 310 includes a slide 324, which is a moveable part 320, and a base 330. The slide 324 is configured to be able to slide into or move into the base 330 as shown in FIG. 5B. The worm structure 340 moves in a vertical direction, along an axis C, to drive the stretching function in the worm-gear bioreactor 310. The stretching function is caused in a direction along an axis D by moving the rack 328 attached to the slide 324 (FIG. 5A). The change of direction form a vertical direction (axis C) to a horizontal direction (axis D) can avoid direct contact with the media when the worm structure 340 in manually or mechanically operated.

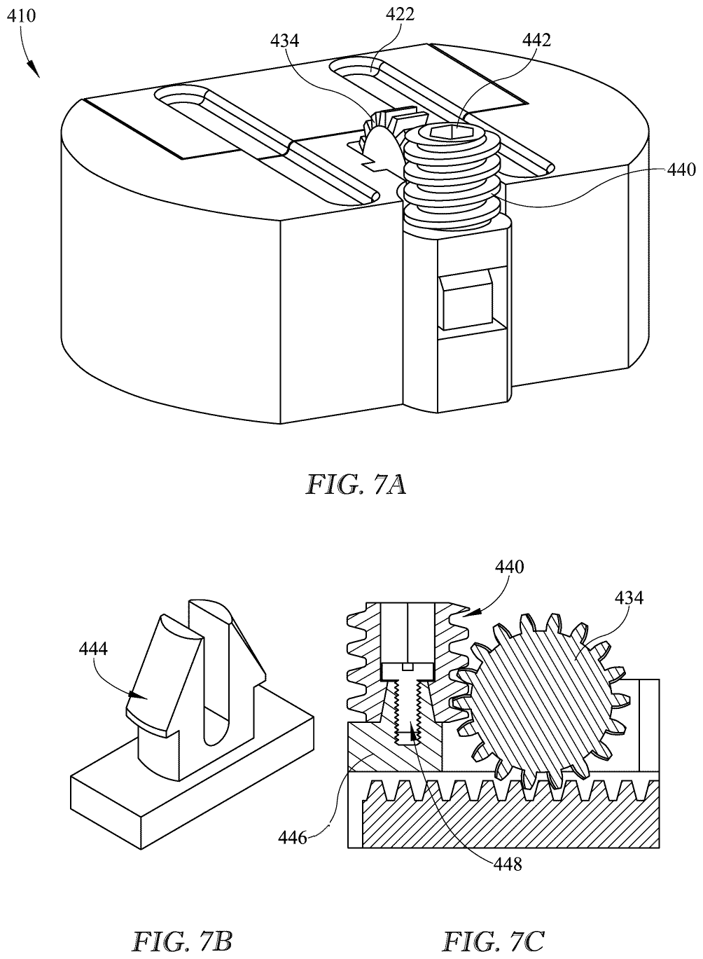

As shown in FIG. 7B, the worm structure 340 can be attached to the base 330 with a snapping structure 444. Since the worm-gear structure comprises a self-locking snapping structure 444, an additional locking structure is not required to lock the slide 324 at the desired position at any time. The worm-gear bioreactor 310 includes rectangular chambers 322 for positioning the grafts 12. The worm-gear bioreactor 310 may include screw holes (not shown) as described earlier.

In other embodiments, the bioreactor 310 may include different mechanisms to translate the vertical motion along the axis C to the horizontal movement of the rack along the axis D. For example, the bioreactor may include a jack system, a link system, a bell crank etc.

FIGS. 6 and 7 illustrate another embodiment of a worm-gear bioreactor 410. The worm-gear bioreactor 410 includes chambers 422 with a cylindrical cross-section to minimize twisting of the grafts 12 when the stretching function is applied (FIG. 6, 7A). Furthermore, a screw connection structure 446 comprising a screw 448 is used to attach a worm structure 440 to a base 430 instead of the snapping structure 444 (FIG. 7C). The meshing of the worm structure 440 and a gear 434 in a driving system 418 enables the sliding and rolling actions of the worm structure 440 and the gear 434 to result in the stretching function. The direction of force transmission from input to output in the worm-gear bioreactor 410 is irreversible. Therefore, the gear 434 cannot drive the worm structure 440 backwards. Thus, the slide 424 will be locked at the exact stretching position without any subsequent displacement.

As shown in FIGS. 7A, the interior of the worm structure 440 is designed as a hollow structure with inner hole 442. The cross-section of the inner hole 442 is a hexagon. Therefore, a hexagon wrench or any other tool can be used to rotate the worm structure 440 from above the worm-gear bioreactor 410, reducing the likelihood of an infection being introduced. In other embodiments, the inner hole 442 can have different cross-sectional shape including but limited to circular, rectangular, and oval. The worm-gear bioreactor 410 can include screw holes 432′ for screws 432 to attach the grafts 12. For example, in the illustrated example, the worm-gear bioreactor 410 includes four screw holes 432′.

During the stretching function of the worm-gear bioreactor 410, the distance the slide 424 is driven outwards does not uniformly increase with the rotation of the worm structure 440. The process from the beginning of the rotation to the end of the rotation comprises a non-linear stretching process and a linear stretching process. This is because of the slack between the worm structure 440 and the gear 434. Therefore, in some embodiments, the slide 424 may be driven out in advance to avoid the nonlinear process. After the slide 424 is driven out, the grafts 12 may be fixed onto the bioreactor 410. During the linear stretching process, the distance that the slide 424 is driven out for every 45° of rotation on the worm structure 440 may range from about 0.4 mm to about 0.5 mm including any distance or range comprised therein, In some embodiments, the distance that the slide 424 is driven out for every 45° of rotation on the worm structure 440 may be about 0.423 mm.

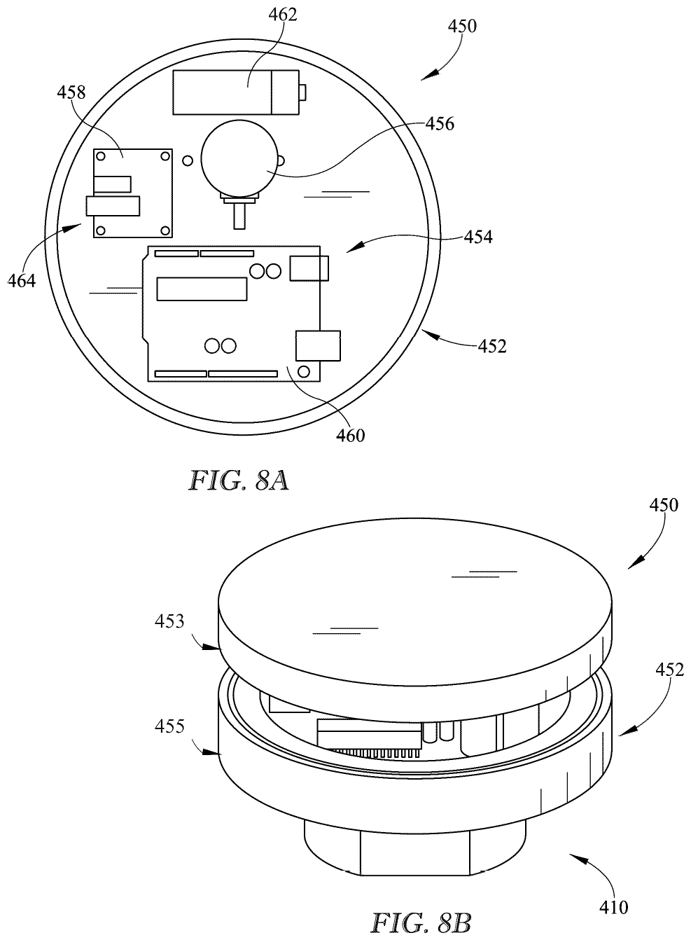

FIGS. 8A and 8B illustrate one embodiment of a bioreactor system 450 comprising a housing 452 and the worm-gear bioreactor 410. The housing 452 includes a housing lid 453 and a housing base 455. The housing base 455 contains electronics 464 used to provide power to the driving system 418 shown in FIG. 6 and engage the graft 12 in a constant and/or continuous stretching function. The housing 452 includes a self-contained power source such as a battery that does not need any external wires or other components. The use of a self-contained power source makes the bioreactor system 10 an autonomous system as the system is not dependent on any external power source. The housing 452 includes a stepper motor system 454 that can rotate the gear 434 (FIG. 6) to move the rack 428 (FIG. 6) to allow for a continuous stretching of graft 12. The housing 452 includes a motor 456 that is driven by a motor driver 458, controlled by an Arduino Uno microcontroller 460, and powered by a 9V battery 462. In one embodiment, a rod connector is connected with a stepper motor shaft by inserting a hex rod into the worm structure 440. Then the microcontroller 460 is connected to the battery 462 before the housing lid 453 is closed over the housing base 455.

In other embodiments, different controllers and/or power sources may be used. The electronics 464 (e.g., motor 456, motor driver 458, Arduino Uno microcontroller 460, 9V battery 462) are housed in a housing 462 to protect them from incubator conditions including media, temperature etc. (FIG. 8B). The electronics 464 can cause an average rack displacement rate of about 2.5 mm/rev to about 5 rev/mm including any value or range comprised therein. In a specific embodiment, the electronics 464 can cause an average rack displacement rate of about 3.2 mm/rev. The stretching function may comprise linear stretching, unidirectional rotation or stretching, compression, multi-axial stretching, and/or oscillatory stretching.

In some embodiments, a wireless adapter can be attached to the microcontroller 460, and the motor output signals from the microcontroller 460 can be controlled wirelessly. For example, a wireless adapter like ESP8266 Wifi module can be connected to the microcontroller 460, which can receive and transmit signals wirelessly. Such a configuration can allow multiple bioreactors 410 to be programmed at the same time. In some embodiments, a load cell and a PID controller can be connected to monitor and control the stress that each graft 12 experiences. Alternatively, each microcontroller 460 can be individually programmed to control the corresponding motor 456 in the bioreactor system 450.

In some embodiments, a micro load cell like CZL639HD can be connected to the bioreactor 410 and the microcontroller 460 in order to measure the mechanical stress. The stress data can be used as feedback to adjust the motor control input through a PID controller. Such a feedback loop can keep the stress consistent during the culture period. A user can monitor the data in real time at a remote location when a wireless adaptor is used.

In some embodiments, the bioreactor 410 may further include one or more sensors including but not limited to a pH sensor and/or a micro-Raman/FTIR probe to monitor the pH of the media and the composition of the graft 12 (e.g., amount of collagen in the grafts 12).

In some embodiments, a rechargeable battery with a charging port can be positioned in the housing 452 or a wireless charging technology can be implemented to charge the bioreactor system 450 in the incubator. In some embodiments, the microcontroller 460 can be connected to buttons positioned on the side of the housing 452. The bioreactor system 450 can be programmed to transmit various pre-programmed signals depending on which button was selected by the user. In some embodiments, an automated culture media circulation can be attached to the bioreactor system 450. Such a configuration can allow fresh culture media to be replenished automatically, hence reducing the labor costs.

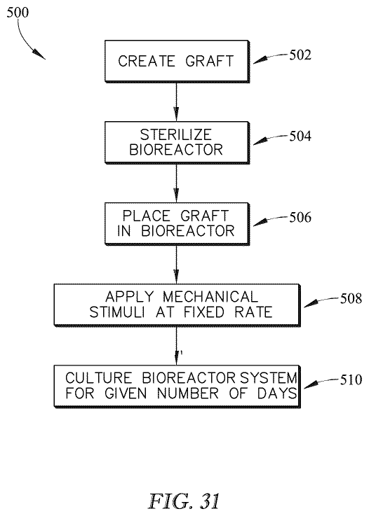

A method 500 of creating tissue-engineered constructs 14 is shown in FIG. 31. The method 500 begins at block 502. At block 502, the method 500 includes creating the grafts 12. Next, in block 504, the method 500 includes sterilizing the bioreactor 10, 110, 210, 310, 410. Next, in block 506, the method 500 includes placing at least one graft 12 in the bioreactor 10, 110, 210, 310, 410. The bioreactor 10, 110, 210, 310, 410 is placed in the container with media 15. The housing 452 is placed above the bioreactor 10, 110, 210, 310, 410. Next, in block 508, the method 500 includes applying a required amount of mechanical stimulation. For example, the electronics 464 of the bioreactor system 2 may be programmed to apply a strain of about 1 mm/day. Next, in block 510, the method 500 includes culturing the grafts 12 in the bioreactor system 2 for a given number of days in an incubator. For example, the grafts 12 may be cultured for about 28 days to form tissue-engineered mensci.

### Example 1: Bone Plugs Extraction

The bone plugs used in this study were prepared according to published methods. Briefly, the bone plugs were extracted from the distal femurs of 1-3-day-old bovids (Gold Medal Packing Inc.) using an electric drill with a 6 mm coring bit. During dissection process, excess tissue around the head and neck of the femur was removed to expose the surface of the trabecular bone, from which bone plugs were extracted. After drilling out 15 mm long cylindrical bone plugs, a stream of high velocity deionized water was used to rinse out residual blood, marrow, and debris from the pore space and keep the white trabecular bone structure. Then bone plugs were washed for 1 hour in PBS with 0.1% (wt/vol) ethylenediaminetetraacetic acid (EDTA) at room temperature. After that, sequential washes in hypotonic buffer (10 mM Trizma base in 100 mL DI water, 0.1 w/v % EDTA) were applied for 1 hour each and at least for three times. Then bone plugs were placed at 4° C. for at least 24 hours. Next, detergent of 10 mM Trizma base with 0.5 w/v % was used for washing bone plugs for at least 3 hours and the bone plugs were room temperature for 24 hours. Finally, the bone plugs were washed using PBS with antibiotics at least seven times until no bubbles were formed.

### Example 2: Rat Tail Collagen Extraction

The collagen used was extracted from rat tails (Pel-Freez Biologicals, Rogers, AZ). The rat tails were immersed in 70% ethanol for 10-20 minutes for thawing. The skin of the rat tail was cut and peeled, and the rat tail tendon was extracted by holding the end of the rat tail and pulling at the tips. After extraction, the collagen was collected into ethanol, the rat tail tendons were dried, and then placed into 0.1% acetic acid. 150 mL of acetic was used per gram of tendon. The collagen was then solubilized for at least 48 hours at 4° C. After that, the collagen solution was placed into 50 mL conical tubes and centrifuged. After being centrifuged at 15000 rpm for 45 minutes at 4° C., the clear supernatant was collected and frozen at below −80° C. for 30 minutes and then lyophilized for 48 hours. Finally, the collagen product was weighed and reconstituted in 0.1% acetic acid solution. The concentration of the collagen was about 30 mg/mL.

### Example 3: FCC Extraction

Fibrochondrocytes (FCC) were extracted from menisci of 1-3-day-old bovids from the knee joints. Menisci were chopped up into 1-2 mm cubes after being dissected from the joint in sterile environment. After that, cubes were placed into PBS with ABAM (Antibiotic Antimycotic Solution, Mediatch, Inc.), washed three times, and incubated for 30 minutes after the third washing. Then the cubes were added into 0.3% collagenase (Worthington Biochemical Corporation, Lakewood, NJ) solution with 1% ABAM for 18 hours of digestion in a 37° C. incubator with spinning. Then, the digested tissue was pipetted into 100 μm cell strainers into conical tubes to isolate FCCs. After spinning down the conical tubes at 2500 rpm for 12 minutes, the supernatant was aspirated and the cell pellet was dispersed with PBS with 1% ABAM. Conical tubes were used for centrifuging at 1000 rpm for nine minutes. Cell concentrations were adjusted to 150*10⁶ cells/mL in media for graft 12 generation.

### Example 4: Graft Generation

The graft 12 comprising a meniscal enthesis construct 12 is composed of bone zone, collagen zone, and an interface between them. The graft 12 is generated from decellularized bone plugs, type I collagen, and fibrochondrocytes prepared as described above. Briefly, marks were made onto a Tygon® tubing to divide zones for collagen and bone. Decellularized bone plugs were placed into the tube at both ends and binder clips were used in avoid of the bone plugs from moving backwards. Holes were cut out at both ends for flowing air during collagen injection. A central hole was cut in the center of the bone zone for injection of collagen. Prepared collagen was mixed with working solution(1 N NaOH, 1× PBS and 10× PBS) and isolated FCCs with media in order to achieve the final collagen mixture with a neutral 7.0 pH, a concentration of 20 mg/mL collagen and 25*10⁶ cells/mL. The mixture was injected into the central hole and the grafts 12 are placed into an incubator under 37° C. for 50 minutes for gelation. After the gelation process, the grafts 12 were removed from the tube into a petri dish with meniscus media and placed in an incubator to equilibrate overnight. Finally, the grafts 12 were clamped onto polysulfone molds with clamps and screws without stretching as a control group, and onto the bioreactor 410 described above as an experimental group to form tissue-engineered constructs 14. Both groups were cultured for the same time periods (2 weeks, 4 weeks). The tissue-engineered constructs 14 were processed for histology analysis and for tensile testing.

### Example 5: Culture Experiments

For the 2-week culture experiment, the graft 12 were fixed onto polysulfone molds without stretching stimulation as the control group. The experimental group grafts 12 were fixed onto stretching bioreactors 410 with axial stretching stimulation. The stretching rate was 2% of initial collagen length (45° rotation on worm) for every 3 days (5 times of stretching in total). The tissue-engineered constructs 14 from both groups were generated with the same decellularized bone plugs and collagen with FCCs under same conditions. The initial length of collagen was 20 mm (Table 1). After culture, all the tissue-engineered constructs 14 from both groups were processed for Picrosirius red staining and Masson's trichrome staining.

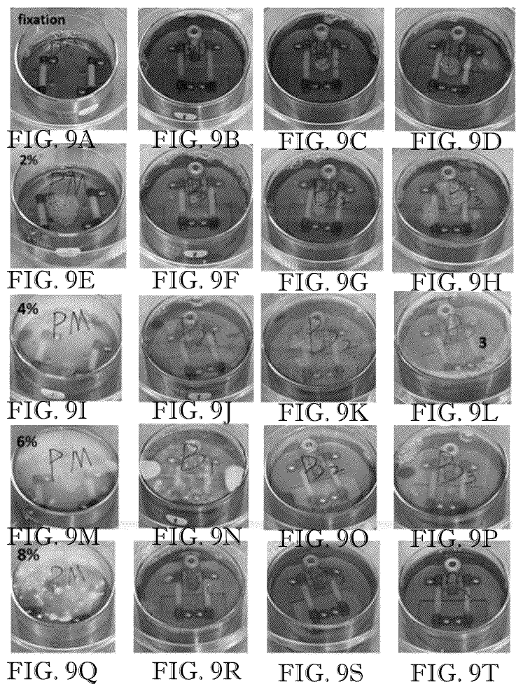

For the 4-week culture experiment, the culture time was changed, and the stretching distance was changed to 8% of initial length of collagen (1.6 mm). Further, the stretching rate was changed from 2% every 3 days to 2% every 6 day. The graft 12 were stretched four times. The other parameters remained unchanged (Table 2). After culture, the collagen length of the tissue-engineered constructs 14 from both groups was measured. Five control group tissue-engineered constructs 14 and four experimental tissue-engineered constructs 14 underwent tensile testing. One control group tissue-engineered constructs 14 and six experimental group tissue-engineered constructs 14 are analyzed with Picrosirius red staining and Masson's trichrome staining. The images of the tissue-engineered constructs 14 placed in containers with media 15 at different time points are shown in FIG. 9A-9T. From top line to bottom, grafts are exposed to 0% (control), 2%, 4%, 6%, 8% stretching respectively.

TABLE 1
2-week culture experiment set

| | Control Group | Experimental Group |
|---|---|---|
| Control | Fixed culture without | Fixed culture with |
| | stretching | stretching |
| Constructs | 20 mm collagen with FCCs + decellularized bone plugs | |
| Reactors | Polysulfone molds | PLA Bioreactors |
| Distance | — | 10% (2 mm) |
| Rate | — | 0.4 mm (2%) every 3 days |

| | |
|---|---|
| Culture time | 14 days |

### Example 6: Histology Analysis

The tissue-engineered constructs 14 were placed into 10% buffered formalin to fix them for 48 hours. After fixing, formalin was washed off with ethanol and the tissue-engineered constructs 14 were longitudinally cut in half to expose their inner structure. Slides of sections were used for Picrosirius red and Masson's trichrome staining. The Picrosirius red staining slides were viewed under brightfield and polarized light with a Nikon Eclipse TE2000-S microscope. The images were taken through a SPOT RT camera.

TABLE 2
4-week culture experiment set

| | Control Group | Experimental Group |
|---|---|---|
| Control | Fixed culture without | Fixed culture with |
| | stretching | stretching |
| Constructs | 20 mm collagen with FCCs + | |
| | decellularized bone plugs | |
| Reactors | Polysulfone molds | PLA Bioreactors |
| Distance | — | 8% (1.6 mm) |
| Rate | — | 0.4 mm (2%) every 6 days |

| | |
|---|---|
| Culture time | 28 days |

### Example 7: Tensile Testing

The 4-week-cultured tissue-engineered constructs 14 were subjected to tensile testing for analysis of mechanical properties. The tensile testing used in this research is based on published methods. Briefly, the tissue-engineered constructs 14 were clamped at both bony ends and vertically fixed onto the testing system (ElectroForce 5500 System, Bose, Eden Prairie, MN). A rate of 1.5 mm/sec was applied to the tissue-engineered constructs 14 until their failure. For mechanical property analysis, several variables were calculated as follow. The ultimate load (UL) was the maximum load that the tissue-engineered constructs 14 can withstand during the testing process. The ultimate tensile stress (UTS) was the maximum stress the tissue-engineered constructs 14 can withstand, and it is the point the tissue-engineered constructs 14 begins to fail. The tensile modulus is the slope of the linear elastic part of stress-strain curve. The toughness is the total area under the stress-strain curve. The strain values were also recorded and analyzed at the onset and at the end of failure.

All the data points in this study are graphed in the form of mean±SD. Data from tensile testing are analyzed with t-testing. Significant difference is determined with p<0.05 and difference existence is determined with 0.05<p<0.1.

### Example 8: Formation of Tissue-Engineered Meniscal Enthesis Constructs

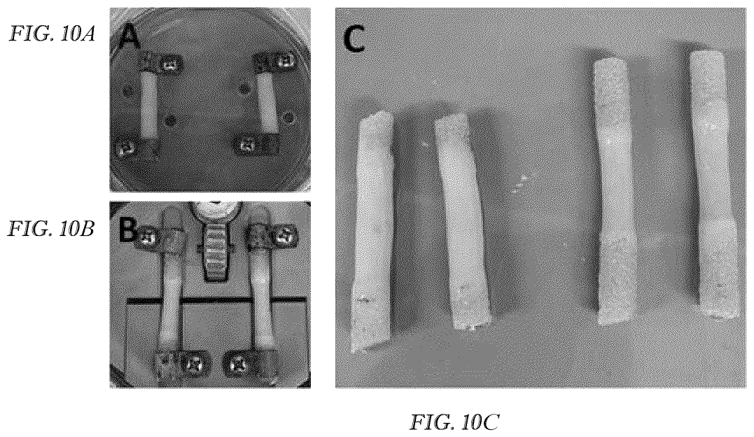

The tissue-engineered constructs 14 (meniscal enthesis constructs) were generated from the grafts 12 on polysulfone molds (control groups) and stretching bioreactors (experimental groups). The culture time for the two experiments was respectively 2 weeks and 4 weeks. Following the same generation method, the bone zone 6 and the collagen zone 8 were recognizable from both the control and the experimental groups. The collagen penetrated into the trabecular bone plugs and formed the interface region 7 between the bone zone 6 and the collagen zone 8. The tissue-engineered constructs 14 from both groups were determined to be stiff enough to withstand conventional physical handling without fracture. In the 2-week culture experiment, the observable necking phenomenon was found in samples from the experimental group compared to the control group (FIG. 10A-C). The collagen length in the control group is about 20 mm, the collagen length in the experimental group is about 22 mm.

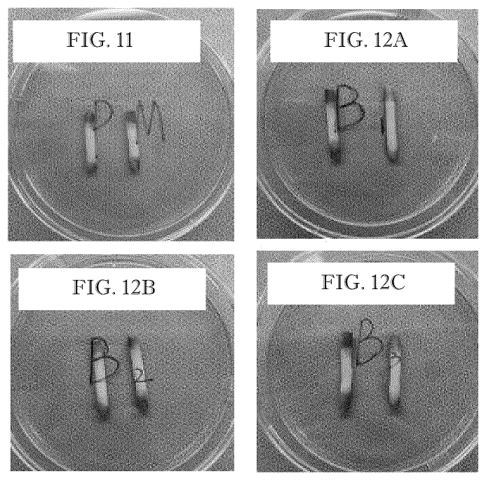

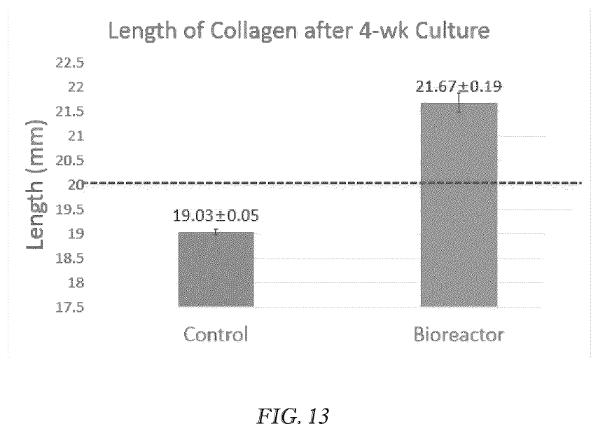

In the 4-week culture experiment, there was no observable necking phenomenon in either group (FIGS. 11, 12A-12C). The length of the tissue-engineered constructs 14 was about 19.03 mm±0.05 mm for the control group and about 21.67 mm±0.19 mm for the experimental group (FIG. 13). On average the collagen length was elongated by about 8.35% after stretching.

### Example 9: Histology Analysis of Tissue-Engineered Meniscal Enthesis Constructs

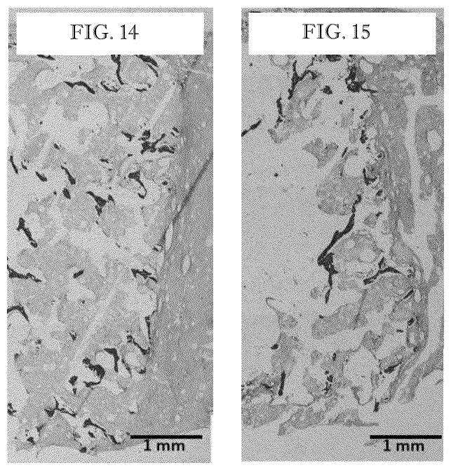

Differences in collagen density can be found by comparing brightfield images of Picrosirius red staining slides at the interface region 7 between the unstretched tissue-engineered constructs 14 and the stretched tissue-engineered constructs 14 after 2 weeks of culture (FIGS. 14, 15) The unstretched tissue-engineered constructs 14 show higher collagen density at the interface compared to stretched tissue-engineered constructs 14. There was an obvious banded gap space between the bone region and the collagen region in stretched tissue-engineered constructs 14, which can be seen as a sign of a partial failure phenomenon at the interface region 7 during the stretching process.

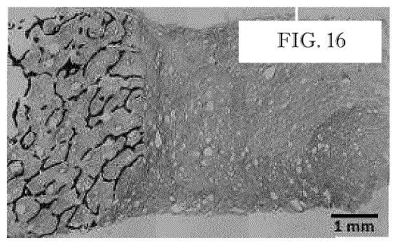

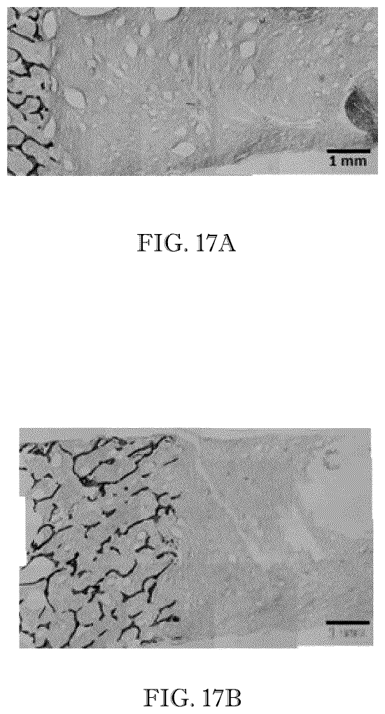

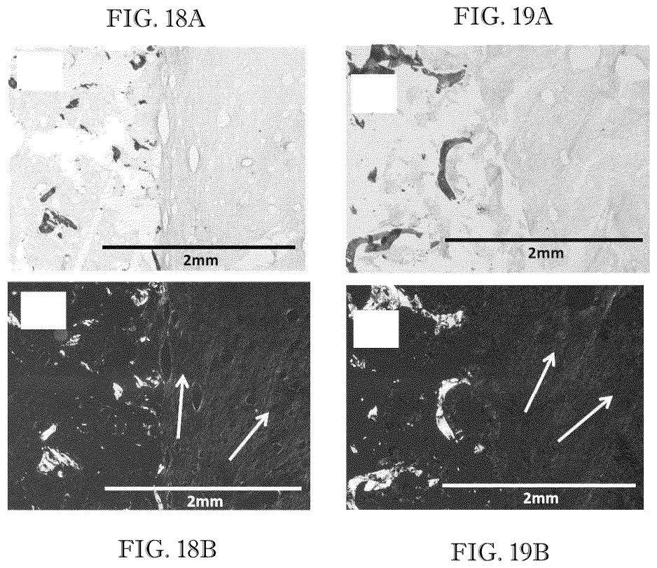

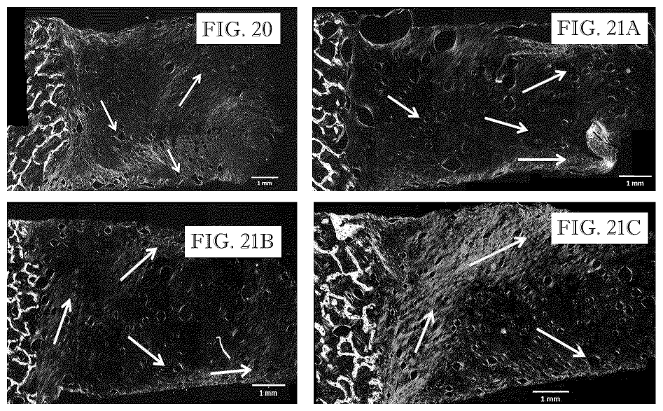

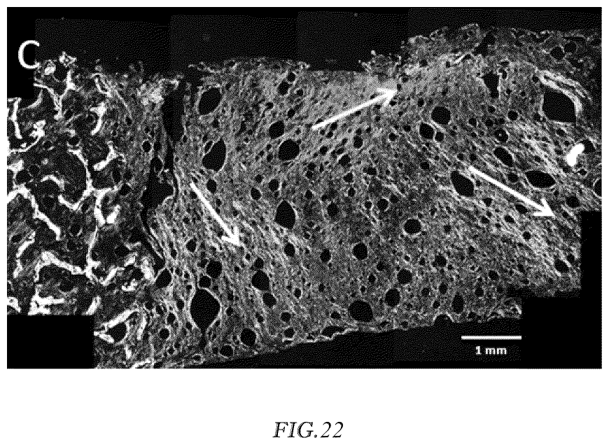

Similar features also appear in the Picrosirius red staining slides of 4-week tissue-engineered constructs 14. The unstretched tissue-engineered constructs 14 (FIG. 16) showed a higher density of collagen and fewer defects at the interface and collagen region than the stretched tissue-engineered constructs 14 (FIGS. 17A and 17B). Even without observable necking phenomenon, the 4-week stretched tissue-engineered constructs 14 showed regions in the collagen with more defects. Similarities of fiber orientations were observed at the interface region 7 in the polarized light images of unstretched and stretched tissue-engineered constructs 14. For both groups, the collagen fiber orientation is mainly parallel to the edge of the interface, which is along the radial direction. The fibers tended to change to a longitudinal direction as the fiber bundles extend to the collagen region (FIGS. 98, 19). This phenomenon was observed both in control groups and experimental groups.

After 4 weeks of culture, differences in collagen fiber organization between unstretched and stretched tissue-engineered constructs 14 were observed (FIGS. 20, 21A-21C). In unstretched tissue-engineered constructs 14, the fiber orientation showed a pattern similar to the 2-week culture experiment: fibers were mainly along the radial direction at the interface and changed to a longitudinal direction in the collagen region. While in the stretched tissue-engineered constructs 14, large areas of fibers along the longitudinal direction were observed at the collagen region, especially at the outer edge of the collagen region. Less radial fiber orientation was found in the stretched tissue-engineered constructs 14 compared to the unstretched tissue-engineered constructs 14. There were more defects in the collagen part of stretched tissue-engineered constructs 14 (FIG. 22).

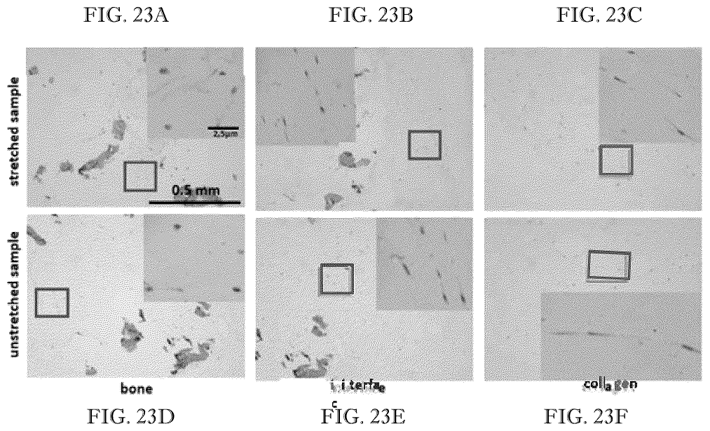

Masson's trichrome staining images demonstrate the cell morphology of FCCs in the tissue-engineered constructs 14. The cells are elongated along the direction of fiber orientations at interface and collagen region, while they remain rounded in shape in the bone region. Both are found in unstretched and stretched tissue-engineered constructs 14, as cells are elongated mainly along the radial direction at the interface region 7 while they change to a longitudinal direction in the collagen region (FIGS. 23A-23F).

### Example 10: Mechanical Analysis of Tissue-Engineered Meniscal Enthesis Constructs

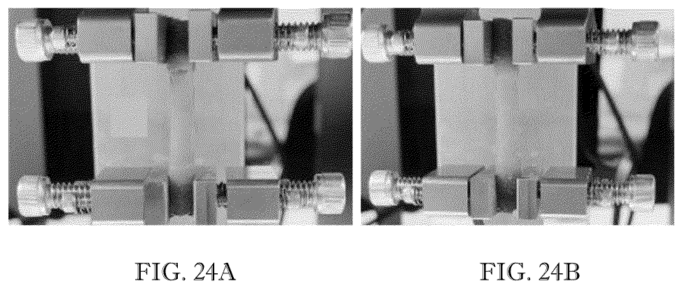

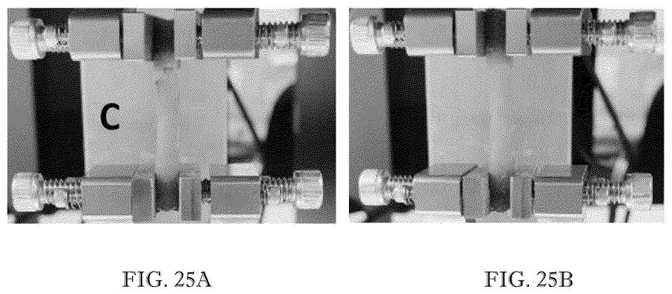

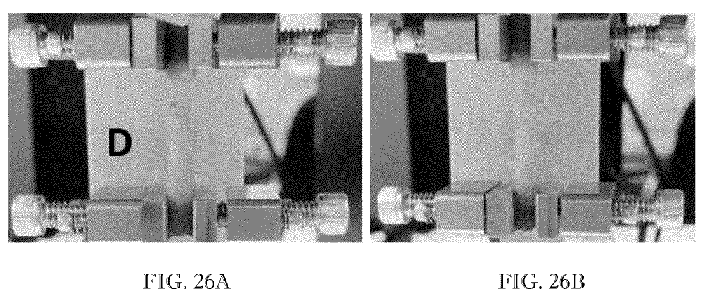

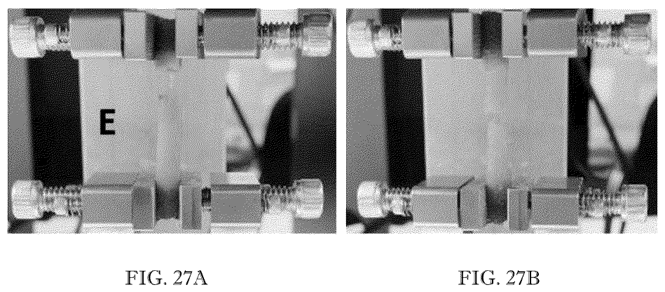

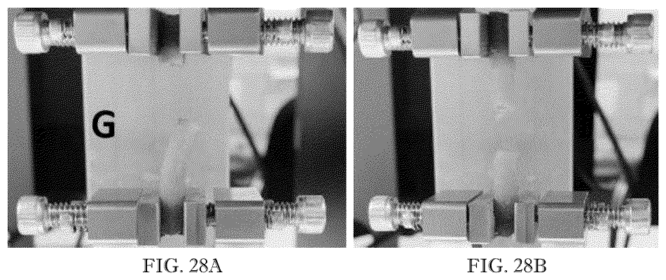

The tissue-engineered constructs 14 from the control groups and the experimental groups showed different features during the stretching to failure process. Still shots of the tensile testing process were taken at the same time points for comparison between the control groups and the experimental groups. Necking was first observed in the control tissue-engineered constructs 14 (FIGS. 24A and 24B), and then stretching failure started in control tissue-engineered constructs 14 at the interface (FIGS. 25A and 25B). When control group tissue-engineered constructs 14 completely failed, necking was observed in the experimental group tissue-engineered constructs 14 (FIGS. 26A and 26B). After that, the failure started in the experimental group tissue-engineered constructs 14 in the middle of the collagen (FIGS. 27A and 27B) until complete failure (FIGS. 28A and 28B).

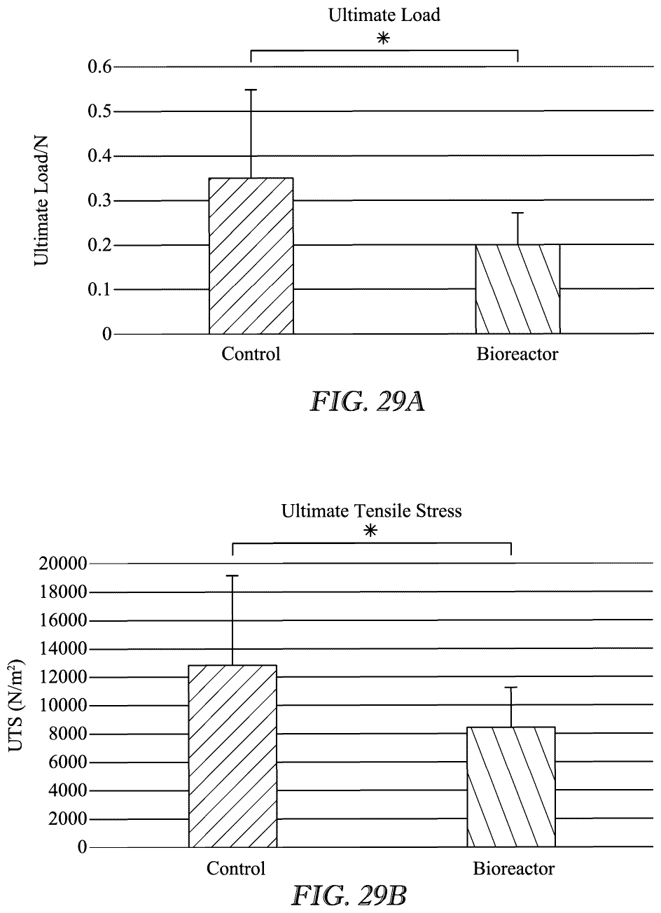

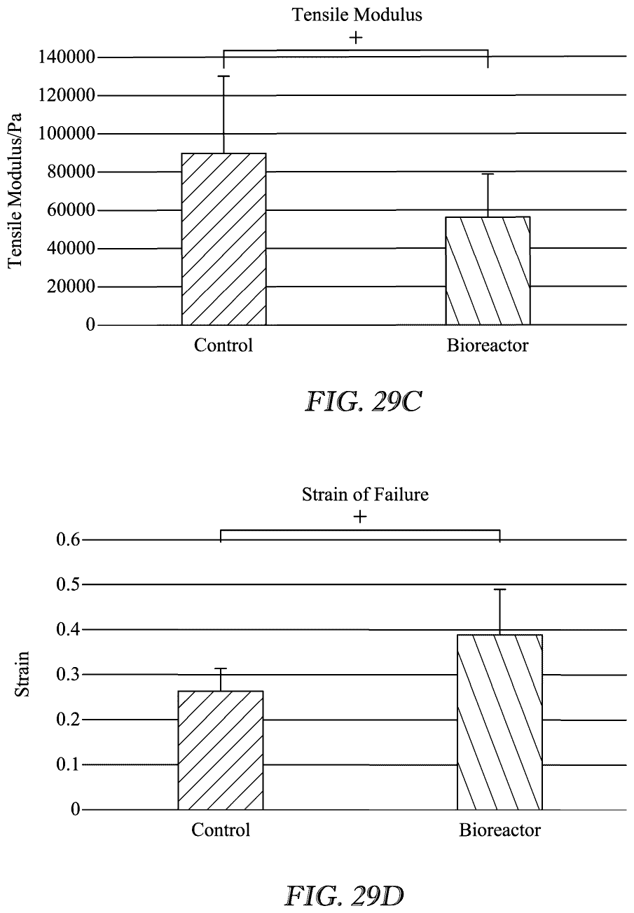

After collecting and analyzing the tensile testing data, the stress-strain curves of the tissue-engineered constructs 14 from both groups were examined. Obvious differences were observed by comparing curves from control groups and experimental groups. After calculation, the values of different mechanical properties are listed in Table 3. Comparison of UL, UTS, tensile modulus, and strain of failure are compared (FIGS. 29A-29D).

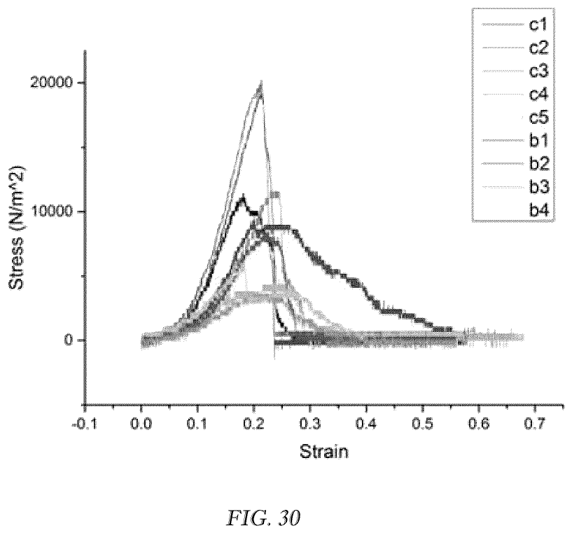

By comparing the stress-strain curves from control groups and experimental groups, it was observed that most of the unstretched tissue-engineered constructs 14 showed higher ultimate load (UL), ultimate tensile stress (UTS), and tensile modulus compared to the stretched tissue-engineered constructs 14. However, the stress-strain curves of stretched tissue-engineered constructs 14 generally showed the feature of higher strain after the onset of failure during tensile testing compared with control tissue-engineered constructs 14 (FIG. 30). As shown in FIG. 30, c1-5 indicate control tissue-engineered construct (black series) and b1-4 (red series) indicate experimental tissue-engineered construct

TABLE 3
Mechanical property data of meniscal enthesis constructs
from control groups and experimental groups.

| | | * UTS | + Tensile | Toughness | + Strain of |
|---|---|---|---|---|---|
| | * UL (N) | (N/m²) | Modulus (Pa) | (J/m³) | Failure |
| Control | 0.356 ± 0.19 | 12986 ± 6218 | 90528 ± 40262 | 1272 ± 345 | 0.266 ± 0.032 |
| Bioreactor | 0.203 ± 0.06 | 8405 ± 2760 | 56249 ± 22762 | 1082 ± 598 | 0.39 ± 0.1 |

* means significant difference (p < 0.05);
+ means difference exists (0.05 < p < 0.1).

The figures provided herein are not necessarily to scale, although a person skilled in the art will recognize instances where the figures are to scale and/or what a typical size is when the drawings are not to scale. Additionally, a number of terms may be used throughout the disclosure interchangeably but will be understood by a person skilled in the art. Further, to the extent features, sides, or steps are described as being “first” or “second,” such numerical ordering is generally arbitrary, and thus such numbering can be interchangeable. Lastly, the present disclosure includes some illustrations and descriptions that include prototypes or bench models. A person skilled in the art will recognize how to rely upon the present disclosure to integrate the techniques, systems, devices, and methods provided for into a product in view of the present disclosures.

What is claimed is:

1. A bioreactor adapted to be placed in a container with liquid media, the bioreactor comprising
a fixed part including a base and a first fixation device configured to fix a first end of a graft in place relative to the base,
a moveable part including a second fixation device configured to fix a second end of the graft in place relative to the movable part, the moveable part mounted to slide relative to the fixed part so as to move the second end of the graft relative to the first end of the graft, and
means for driving the movable part to slide relative to the fixed part such that the second fixation device moves toward and away from the first fixation device to decrease or increase tension in the graft to thereby mechanically stimulate the graft, wherein the means for driving includes a mechanical input that extends upwardly to a point above the first fixation device and the second fixation device so that sliding of the movable part can be effected without undue disturbance of liquid media at a level above the first and the second fixation devices such that the graft is submerged but also at a level below at least a top surface of the mechanical input.

2. The bioreactor of claim 1, wherein the means for driving includes a drive system in which the mechanical input is provided by a worm structure that rotates about a vertical axis to cause a gear located in the fixed part to move a rack coupled to the moveable part, and wherein the moveable part is mounted to move along a horizontal axis relative to the fixed part.

3. The bioreactor of claim 2, wherein the worm structure is attached to the base with a screw configured to drive friction between the worm structure and adjacent components that resists reverse motion of the worm structure in response to tension developed in the graft.

4. The bioreactor of claim 1, any other suitable claim, or any other suitable combination of claims, wherein at least one of the first fixation device and the second fixation device is provided by a clamp that overlies a portion of the fixed part or the movable part.

5. The bioreactor of claim 4, wherein the clamp includes an engagement panel that extends over portion of the fixed part or the movable part and a screw adapted to extend through the engagement panel and into one of the fixed part or the movable part.

7. The bioreactor of claim 2, wherein the bioreactor is incorporated into a bioreactor system along with a housing that includes a self-contained power source for powering the drive system.

8. The bioreactor of claim 7, wherein the housing includes a motor configured to rotate the mechanical input of the drive system.

9. The bioreactor of claim 8, wherein the power source and the motor are controlled by a microcontroller and the micro controller is configured to adjust input to the mechanical input of the drive system to maintain preselected mechanically stimulation of the graft.

10. The bioreactor of claim 9, wherein the power source is a battery.

11. A method of creating a tissue-engineered construct comprising
positioning a graft comprising a bone region, a collagen region, and an interface region in a bioreactor, wherein the bioreactor includes a
a fixed part with a base positioned in media,
a moveable part having a chamber for positioning the graft and wherein the moveable part can slide into the fixed part, and
a drive system including a worm structure that moves along a vertical axis to cause a gear located in the fixed part to move a rack located in the moveable part, wherein the moveable part moves along a horizontal axis away from the fixed part;
exposing the graft to a continuous mechanical stimulation caused by motion of the drive system; and
placing the bioreactor in an incubator for a period of time.

12. The method of claim 11, wherein the graft is positioned in an indented chamber defined in part by the fixed part and in part by the movable part.

13. The method of claim 11, wherein the drive system is powered by a self-contained power source located in a housing that can be selectively attached to the bioreactor.

14. The method of claim 13, wherein the mechanical stimulation is caused by a motor rotating the gear to move the rack along the horizontal axis, and wherein the motor is located in the housing

15. The method claim 14, wherein a displacement rate of the rack is about 3.2 mm/rev.

16. The method of claim 11, wherein the tissue-engineered construct is a meniscal enthesis construct.

17. The method of claim 11, wherein exposing the graft to a continuous mechanical stimulation increases integration between the bone region and the collagen region at the interface region.

18. The method of claim 11, further comprising submerging the fixed part, the movable part, and a portion of the drive system in liquid media such that an unsubmerged portion of the drive system is available outside the liquid media to accept mechanical input to the drive system without requiring undue disturbance of the liquid media.

19. A modular bioreactor system for creating a tissue-engineered construct, the bioreactor system comprising
a bioreactor including
a fixed part including a base and a first fixation device configured to fix a first end of a graft in place relative to the base,
a moveable part including a second fixation device configured to fix a second end of the graft in place relative to the movable part, the moveable part mounted to slide relative to the fixed part so as to move the second end of the graft relative to the first end of the graft, and
a driving system including a worm structure that moves along a vertical axis to cause a gear located in the fixed part to move a rack located in the moveable part, wherein the moveable part moves along a horizontal axis away from the fixed part, and
a housing including a motor that can rotate the worm structure to move the rack and thereby mechanically stimulate the graft, a microcontroller to control the motor, and a self-contained power source for powering the driving system.

20. The bioreactor system of claim 19, wherein the microcontroller can receive and transmit signals wirelessly.
